(19) United States
(12) Patent Application Publication
(10) Pub. No.: US 2020/0071854 A1
(43) Pub. Date: Mar. 5, 2020

(54) MULTICOMPONENT FILAMENTS AND ARTICLES THEREOF

(71) Applicant: 3M INNOVATIVE PROPERTIES COMPANY, SAINT PAUL, MN (US)

(72) Inventors: HAOMING RONG, WOODBURY, MN (US); MIKHAIL A. BELKIN, MINNEAPOLIS, MN (US); MATTHEW T. SCHOLZ, WOODBURY, MN (US); RANJANI V. PARTHASARATHY, WOODBURY, MN (US); HANNAH C. COHEN, SAINT PAUL, MN (US); AMANDA C. ENGLER, WOODBURY, MN (US)

(73) Assignee: 3M INNOVATIVE PROPERTIES COMPANY, SAINT PAUL, MN (US)

(21) Appl. No.: 16/468,083

(22) PCT Filed: Dec. 7, 2017
(86) PCT No.: PCT/US2017/065020

§ 371 Date: Jun. 10, 2019

Related U.S. Application Data
(60) Provisional application No. 62/433,637, filed on Dec. 13, 2016.

(51) Int. Cl. D01F 8/06; D01F 8/16; A61F 13/36; A61L 15/22; A61L 15/52

(52) U.S. Cl. CPC D01F 8/06; D01F 8/16; A61F 13/36; A61L 15/225; A61F 2013/15512; D10B 2401/022; D10B 2401/021; D10B 2509/02; A61L 15/52

(57) ABSTRACT

Aspects of the present disclosure relate to a multicomponent filament and articles thereof. The multicomponent filament comprises at least a first component and a second component. The first component includes a thermoplastic polymer. The second component includes a hydrophilic thermoplastic polymer comprising 65% (w/w) to 90% (w/w) (inclusive) hydrophilic segments. The first component is capable of forming a continuous filament with the second component.

### BACKGROUND

Polymer filaments are useful in a variety of products including medical and hygiene products, carpets and floor coverings, apparel and household textiles, filtering media, agro- and geotextiles, automotive interior, filler for sleeping bags, comforters, pillows, and cushions, cleaning wipes, abrasive articles, and numerous other products.

Filaments used in medical articles can have a balance of properties such as coefficient of friction, absorbency, mechanical strength, or a combination thereof. For example, an article made from filaments and used in surgical applications, such as a laparotomy sponge, needs to balance the “slip and grip” (e.g., coefficient of friction) properties of the article. High grip fibers (such as cotton) may have issues when applied to soft tissue areas because the article can abrade the soft tissue area. Conversely, high slip filaments (such as hydrogels) may have issues when applied to soft tissue areas because the article can be too slippery to manipulate and/or hold the soft tissue areas.

### SUMMARY

Aspects of the present disclosure relate to a multicomponent filament having a balance of slip and grip properties. The multicomponent filament comprises at least a first component and a second component. The first component includes a thermoplastic polymer. The second component includes a hydrophilic thermoplastic polymer comprising 65% (w/w) to 90% (w/w) (inclusive) hydrophilic segments. The first component is capable of forming a continuous filament with the second component.

Various aspects of the present disclosure also relate to a yarn comprising the second component. Knitted and woven articles comprising the yarn are also provided.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

As used herein, “a,” “an,” “the,” “at least one,” and “one or more” are used interchangeably. Thus, for example, “a” fiber can be interpreted to mean “one or more” fibers.

The term “and/or” means one or all of the listed elements or a combination of any two or more of the listed elements.

Also herein, the recitations of numerical ranges by endpoints include all numbers subsumed within that range (e.g., 1 to 5 includes 1, 1.5, 2, 2.75, 3, 3.80, 4, 5, etc.).

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

Additional details of these and other embodiments are set forth in the accompanying drawings and the description below. Other features, objects and advantages will become apparent from the description and drawings, and from the claims.

### BRIEF DESCRIPTION OF THE DRAWINGS

FIG. 1A-1D illustrates a schematic cross-sections of four exemplary multicomponent filaments, according to various embodiments.

FIG. 2 illustrates a block diagram of a system for processing a multicomponent yarn, according to various embodiments.

FIG. 3 illustrates a scanning electron microscope (SEM) image of a yarn comprising multicomponent filaments, according to various embodiments.

FIG. 4 illustrates a perspective view of a yarn, according to various embodiments.

FIG. 5 illustrates a knitted article comprising a first yarn and a second yarn using a stockinette stitch, according to various embodiments.

FIG. 6 illustrates a woven article comprising a hydrophilic fiber and a secondary fiber, according to various embodiments.

While the above-identified drawing figures set forth several embodiments of the disclosure, other embodiments are also contemplated, as noted in the discussion. In all cases, this disclosure presents the invention by way of representation and not limitation. It should be understood that numerous other modifications and embodiments can be devised by those skilled in the art, which fall within the scope and spirit of the principles of the invention. The figures may not be drawn to scale.

### DETAILED DESCRIPTION

Aspects of the present disclosure relate to multicomponent filaments, yarns, and articles that absorb aqueous liquids. The present disclosure relates to a multicomponent fiber having a balance of slip and grip properties. Thus, the articles comprising the multicomponent fiber are particularly useful for contact with soft tissue areas.

Before any embodiments of the present disclosure are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “connected” and “coupled” and variations thereof are used broadly and encompass both direct and indirect connections and couplings. Further, “connected” and “coupled” are not restricted to physical or mechanical connections or couplings. It is to be understood that other embodiments may be utilized and structural or logical changes may be made without departing from the scope of the present disclosure. Furthermore, terms such as “front,” “rear,” “top,” “bottom,” and the like are only used to describe elements as they relate to one another, but are in no way meant to recite specific orientations of the apparatus, to indicate or imply necessary or required orientations of the apparatus, or to specify how the invention described herein will be used, mounted, displayed, or positioned in use.

“Melt flow index” is a measure of the ease of flow of the melt of a thermoplastic polymer. The melt flow index is measured using the method of American Society for Testing and Materials (ASTM) D1238-04 for the polymer type unless otherwise noted.

“Spinnable” means able to be spun and collected as a continuous filament. As used herein, the term spinning refers to extrusion and solidification of potentially endless filaments, and does not refer to a process where short pieces of staple fibers are twisted into a yarn.

“Yam” means a grouping of filaments or fibers. The term yarn as used herein is not necessarily twisted.

“Tow” means a grouping of filaments and can be used interchangeably with yarn.

“Knitted” means formed from a yarn using a technique that creates multiple interlocking loops of yarn from a continuous yarn. Knitted can also refer to an article formed from a continuous first yarn (which can also refer to lengths of yarn tied end to end) and placing a second yarn through the first yarn.

“Nonwoven” refers to a fabric-like material made from long fibers and bonded together by chemical, mechanical, heat, or solvent treatment.

“Woven” means formed from interlacing two sets of yarns at right angles to each other. The weaving may be performed by using a loom.

“Staple Fiber” refers to fibers that have determinate length, generally between 5-200 mm and a fiber diameter of about 0.5 to 100 microns. Synthetic staple fibers are generally cut to a specific length. Natural staple fibers typically have a range of lengths in each sample. Staple fibers may have a crimp imparted to them.

A multicomponent filament described in the present disclosure has at least a first component and a second component.

The first component can provide structural functionality of a multicomponent filament. Certain properties, such as elasticity, strength, and durability, are desired properties of the first component. For example, the first component can have a tensile strength of 5-50 Mpa using ASTM D638. The first component comprises a thermoplastic polymer that contributes to the structural characteristics of the multicomponent filament. For example, the thermoplastic polymer can improve the wet or dry tensile strength of the resulting multicomponent filament. The thermoplastic polymer is also capable of being extruded. The thermoplastic polymer can be largely hydrophobic and relatively elastic.

Generally, the thermoplastic polymer can have a melt-flow index from 10 g/10 min to 100 g/10 min at 190 degrees C. (inclusive), preferably from 20 g/10 min to 40 g/10 min at 190 degrees C. (inclusive).

The thermoplastic polymer can include a variety of classes, such as styrenic block copolymers, thermoplastic olefins, elastomeric alloys (e.g., elastomeric thermoplastic acrylate block copolymers such as PMMA-polybutylacrylate-PMMA commercially available under the trade designation Kurarity from Kuraray Company, Ltd., Okayama, Japan), thermoplastic polyurethanes (TPUs), thermoplastic copolyesters, and thermoplastic polyamides. The first component can be made of one or more thermoplastic polymers. Thermoplastic copolyesters, thermoplastic polyurethanes and thermoplastic olefins can be particularly useful in the first component of the multicomponent filament because of resistance to pilling.

Thermoplastic copolyesters can be useful as a first component. Particularly useful are thermoplastic aliphatic polyesters which may further include polylactic acid, polycaprolactone, and other biodegradable polymers. A polylactic acid may be an L-lactic acid or D-lactic acid homopolymer; or, it may be a copolymer, such as one that contains L-lactic acid monomer units and D-lactic acid monomer units. (In such polymers, a homopolymer or copolymer designation will be a “stereo” designation based on the tacticity of the monomer units rather than on the chemical composition.) Again, such monomer units may be derived from the incorporation into the copolymer chain of L-lactic acid, D-lactic acid, L-lactide, D-lactide, meso-lactide, and so on. In some embodiments, a polylactic acid may be an L-D copolymer comprised predominately of L-lactic acid monomer units along with a small amount of D-lactic acid monomer units (which may e.g. improve the melt-processability of the polymer). In various embodiments, a polylactic acid copolymer may comprise at least about 85, 90, 95, 96, 97, 98, 99, 99.5, or 99.7 wt. % L-lactic acid monomer units. In further embodiments, a polylactic acid copolymer may comprise at most about 15, 10, 5, 4, 3, 2, 1, 0.5, or 0.3 weight % D-lactic acid monomer units.

In some embodiments, substantially all (i.e., 99.5 wt. % or greater) of the polylactic acid content of the first component (and/or of the entire polymeric content of the filaments) may be provided by polylactic acid (stereo)copolymer; e.g., a copolymer comprised predominately of L-lactic acid monomer units along with a small amount of D-lactic acid monomer units. (In specific embodiments, substantially all of the polylactic acid content of the filaments may be in the form of L-lactic acid homopolymer.) In other embodiments, an additional, small amount of polylactic acid consisting of D-lactic acid (stereo) homopolymer may be present. Adding such an additional amount of D-lactic acid homopolymer (e.g. as a physical blend, e.g. as a melt additive during extrusion) may in some cases enhance certain properties (e.g. melt-processability, nucleation rate, and so on) of the polylactic acid materials. Thus, in various embodiments, a polylactic acid used. e.g., in meltspinning may comprise at least about 0.5, 1, 2, 3, 5, or 8 wt. % of a D-lactic acid homopolymer additive. In further embodiments, a polylactic acid material may comprise at most about 15, 10, 8, 5, 3, 2, 1, or 0.5 wt. % of a D-lactic acid homopolymer. (In such cases, the balance of the polylactic acid filament-forming material may be an L-D stereocopolymer as noted above.)

In some embodiments, at least some polylactic acid that is present in the first component may be a (compositional) copolymer that comprises one or more additional (non-lactic acid) monomer units. Such monomer units might include e.g. glycolic acid, hydroxypropionic acid, hydroxybutyric acid, and the like. In various embodiments, lactic acid monomer units (whether L or D, and being derived from whatever source) may make up at least about 80, 85, 90, 95, 97, 99, or 99.5 weight % of the polylactic acid filaments.

Melt-processable (filament-forming) polylactic acid polymer materials (e.g., L-D copolymers) are commercially available e.g. from Natureworks LLC of Minnetonka, Minn., under the trade designations INGEO 6100D, 6202D, and 6260D. Melt-processable polylactic acid polymer materials (e.g., D-lactic acid homopolymers) are available, e.g., from Synbra Technologies. The Netherlands, under the trade designation SYNTERRA PDLA 1010. Many other potentially suitable polylactic acid materials are also available.

Thermoplastic polyurethanes (TPUs) can be useful as a first component because of high elasticity. The TPU polymer can be characterized by block copolymers composed of soft and hard segments. Modification of the soft segments can result in a TPU that falls into two groups, polyester-based TPU and polyether-based TPUs (discussed herein). Of particular interest as a first component is the polyester-based TPU due to high abrasion resistance and adhesion strength when compared to polyether-based TPUs. A non-limiting example of a polyester-based thermoplastic polyurethane can be obtained commercially under the trade designation IROGRAN (model PS 440-200) sold by the Huntsman Corporation (The Woodlands, Tex.). Although polyester-based TPU resins are referenced, polyether TPU resins can also be used such as those commercially available under the trade designation Estane from B.F. Goodrich Company (Cleveland, Ohio).

In general, thermoplastic olefins useful in the composition of the multicomponent filament include polymers and copolymers derived from one or more olefinic monomers of the general formula CH₂═CHR″, wherein R″ is hydrogen or C1-18 alkyl. Examples of such olefinic monomers include propylene, ethylene, 1-butene, 1-hexene, 1-octene, 1-decene, 4-methyl-1-pentene, and 1-octadecene, with ethylene being generally preferred. Representative examples of polyolefins derived from such olefinic monomers include polyethylene, polypropylene, polybutene-1, poly(3-methylbutene), poly(4-methylpentene) and copolymers of olefinic monomers discussed herein.

The thermoplastic olefins can optionally comprise a copolymer derived from an olefinic monomer and one or more further comonomers that are copolymerizable with the olefinic monomer. These comonomers can be present in the thermoplastic olefin in an amount in the range from about 0.1 to 10 wt-% based on the total weight of the thermoplastic olefin. Useful such comonomers include, for example, vinyl ester monomers such as vinyl acetate. C1-C18 acrylates such as methyl acrylate, ethyl acrylate, 2ethylhexyl acrylate and the like, vinyl propionate, vinyl butyrate, vinyl chloroacetate, vinyl chloropropionate; acrylic and alpha-alkyl acrylic acid monomers, and their alkyl esters, amides, and nitriles such as acrylic acid, methacrylic acid, ethacrylic acid, methyl acrylate, ethyl acrylate, N,N-dimethyl acrylamide, methacrylamide, acrylonitrile; vinyl aryl monomers such as styrene, o-methoxystyrene, p-methoxystyrene, and vinyl naphthalene; vinyl and vinylidene halide monomers such as vinyl chloride, vinylidene chloride, and vinylidene bromide; alkyl ester monomers of maleic and fumaric acid such as dimethyl maleate, and diethyl maleate; vinyl alkyl ether monomers such as vinyl methyl ether, vinyl ethyl ether, vinyl isobutyl ether, and 2-chloroethyl vinyl ether; vinyl pyridine monomers; N-vinyl carbazole monomers, and N-vinyl pyrrolidine monomers.

The thermoplastic olefin can also contain a metallic salt form of an acid modified polyolefin such as ethylene acrylic acid, or a blend thereof, which contains free carboxylic acid groups. Illustrative of the metals which can be used to provide the salts of said carboxylic acid polymers are the one, two and three valence metals such as sodium, lithium, potassium, calcium, magnesium, aluminum, barium, zinc, zirconium, beryllium, iron, nickel and cobalt.

Suitable thermoplastic olefins are melt-processable or extrudable and include homopolymers and copolymers of polypropylene, homopolymers and copolymers of polyethylene, and homopolymers and copolymers of poly-1-butene. In one aspect, the thermoplastic olefin of the first component is a homopolymer or copolymer of polypropylene.

The thermoplastic olefins can comprise a variety of commercially available materials such as polypropylene, polyethylene (such as linear low density polyethylene or linear low density polyethylene), block copolymer polypropylene, etc. Non-limiting examples of a thermoplastic olefin (such as metallocene polyolefins) suitable to form the multicomponent filament include polymers under the trade designation Affinity and Engage (model 8402) sold by the Dow Chemical Company (Midland, Mich.), and polymers under the trade designation DNDB-1077 NT 7 sold by the Dow Chemical Company (Midland, Mich.).

A thermoplastic olefin can also include blends of the mentioned polyolefins with other polyolefins, or multi-layered structures of two or more of the same or different polyolefins. In addition, they may contain conventional adjuvants such as antioxidants, light stabilizers, acid neutralizers, fillers, antimicrobials, surfactants, antiblocking agents, pigments, primers and other adhesion promoting agents. It may be particularly beneficial for medical applications to incorporate the antimicrobials and enhancers discussed in U.S. Pat. No. 7,879,746, incorporated herein by reference. It may be particularly beneficial for certain applications to incorporate surfactants discussed in US Patent Publication No. 20120077886, incorporated herein by reference.

The first component can also include materials in addition to thermoplastic olefins, such as monomers, oligomers, polymers, or even natural materials (e.g., cotton, rayon, or rubber). For example, the first component can include exemplary monomers such as lactide, glycolide, and the like, and combinations thereof. Exemplary oligomers useful in the presently disclosed second material include oligomers of lactic acids, oligomers of glycolic acids, co-oligomers of lactic and glycolic acids. In addition, these exemplary co-oligomers may be made with other functional monomers, such as, for example, [epsilon]-caprolactone, 1,5-dioxepan-2-one, trimethylene carbonate, or other suitable monomers to obtain an oligomer with a degradation rate different than that of the first material. Exemplary materials useful in the first component include oligomeric co-polymers of lactic and glycolic acids, amine terminated polypropylene glycol, polylactic acid, and combinations thereof. The first component can have a variety of acidity levels.

In at least one embodiment, the first component can be a styrenic block copolymers. Styrenic block copolymers can possess physical and mechanical properties characteristic of filled vulcanized elastomers. Examples of styrenic block copolymers can include styrene/isoprene/styrene and styrene/butadiene/styrene. Further examples can include those available under the model number G1643, and MD6705 by the Kraton Performance Polymer Company (Houston, Tex.).

The second component has hydrophilic characteristics. The second component can comprise at least a hydrophilic thermoplastic polymer that generally comprises hydrophilic polymer/oligomer segments either in the main polymer chain or pendant to the polymer chain. Presently preferred hydrophilic polymers include hydrophilic polymer segments in the main polymer chain. The second component can also comprise a second polymer as discussed herein.

Hydrophilic thermoplastic polymers can refer to polymers that are water soluble which means the polymers can form a visibly transparent homogenous solution in deionized water at 1% wt/wt polymer in water at 25 degrees C. More preferably the hydrophilic polymers can form a visibly transparent homogenous solution in deionized water at 5% wt/wt polymer in water at 25 degrees C. To test solubility the polymer is typically added to deionize water and heated with stirring to 80 degrees C. for 4 hrs and allowed to cool with stirring for 8 hrs. Solutions that are particularly viscous may form trapped air bubbles which can be removed by centrifugation at a speed sufficient to degas the sample but not allow settling of undissolved polymer. Hydrophilic polymers can also refer to polymers that are water swellable and can be capable of absorbing at least 200/%, at least 400%, or at least 1000% of its weight in water to form a swollen gel. An exemplary thermoplastic hydrophilic polymer can be an aliphatic thermoplastic polyurethane polymer such as those having at least about 60% (w/w) hydrophilic segments of hydrophilic polymers.

Exemplary hydrophilic segments include polyethylene glycol groups, polypropylene glycol groups, polybutylene oxide groups, random poly(C₂-C₄)alkylene oxide groups, polyester groups (such as those derived from hydrophilic polyesters (e.g., polyPEG400 succinate)), amine-terminated polyester groups, amine-terminated polyamide groups (such as those derived from amine-terminated unsaturated polyamides disclosed at Patel in Rasayan J. Chem, at http://rasayanjournal.co.in/vol-3/issue-1/20.pdf), polyester-amide groups (such as those derived from hydrophilic polyamides (e.g., polyPEG400diamine succinate)), polycarbonate groups, or combinations thereof. In at least one embodiment, the hydrophilic thermoplastic polymer comprises at least 50%, preferably at least 60%, at least 70%, or at least 80% polyalkylene oxide by weight. The hydrophilic thermoplastic polymer comprises no greater than 90%, no greater than 85% polyalkylene oxide by weight, or any combination with the aforementioned polyalkylene oxide concentration. Although reference is made specifically to polyethylene oxide throughout this disclosure, various hydrophilic segments such as other hydrophilic polyalkylene oxides (described further herein) can be used.

In at least one embodiment, a thermoplastic polymer has one or more hydrophilic segments to make the thermoplastic polymer overall hydrophilic. The hydrophilic segments can be connected through amide, oxamide, ester, urea and/or urethane linkages. In at least one embodiment, the hydrophilic thermoplastic polymer is an aliphatic thermoplastic polyurethane (TPU) polymer (such as a polyether-based or a polyester-based TPU polymer) and has at least about 60% (w/w) hydrophilic segments. Even though reference is made to polyether-based TPU polymers through this disclosure, polyester-based TPU polymers can also be utilized, e.g., by incorporating a small portion of a polyester polyol, such as a polyethylene succinate (hydrophilic).

In at least one embodiment, the second component comprises an aliphatic polyether thermoplastic polyurethane polymer having no greater than about 85% (w/w) polyalkylene oxide. In at least one embodiment, the second component comprises an aliphatic polyether thermoplastic polyurethane (TPU) polymer having at least about 65% (w/w) polyalkylene oxide. For example, the aliphatic polyether thermoplastic can have 65% (w/w) to 90% (w/w), 70% (w/w) to 90% (w/w), 80% (w/w) to 90% (w/w) or even 80% (w/w) to 85% (w/w) polyalkylene oxide.

Aliphatic polyether TPU polymers are known in the art. Aliphatic polyether TPU polymers that are suitable to make multicomponent filaments of the present disclosure include polymers that comprise block subunits of polyalkylene oxides. Suitable polyalkylene oxides include, for example, polyethylene oxide (PEO) (i.e., polyethylene glycol), polypropylene oxide (PPO), polytetramethylene oxide, or mixtures thereof. In at least one embodiment, the polymer used to form a nonwoven fabric is a medical grade TPU polymer. A nonlimiting example of a medical grade TPU polymer suitable to form multicomponent filaments of the present disclosure is the trade designated TECOPHILIC hydrogel TPU (Part number TG-2000 or TG-500) or trade designated TECOFLEX (Part-number EG80A) sold by The Lubrizol Corporation (Wickliffe, Ohio). Table 1 illustrates an estimated composition of sample TPU polymers.

TABLE 1
Composition of sample TPU polymers.

| | | mol % ethylene | | |
|---|---|---|---|---|
| | mol % MDCA | oxide | diol n | diol MW |
| TG-500 | 6.5 | 93.5 | 14.38 | 651 |
| TG-2000 | 3 | 97 (THF) | 32.33 | 1441 |
| EG80A | 9.9 | 90 | 9.09 | 673 |

In at least one embodiment, the TPU polymer TG-2000 can have polyalkylene oxide segments (e.g., PEG diol) between PEG 1000 and PEG 2000, or a mixture of the two. The TG-2000 can be based on a PEG diol with a Mw of about 1500. In at least one embodiment, the polyalkylene oxide in the TPU polymer (e.g., TG-500) can be PEG 600 or a polyalkylene oxide having an average molecular weight of 635. In at least one embodiment, the block subunits of polyalkylene oxide in the TPU polymer can have a formula weight of at least about 1,000, 2000, 3000, 4000, and 5000 daltons and preferably is less than about 20,000, 18,000, 16,000, or 14,000 daltons. In at least one embodiment, the block subunits of polyalkylene oxide in the TPU polymer can have a formula weight of about 6,000 daltons. In at least one embodiment, the block subunits of polyalkylene oxide in the TPU polymer can have a formula weight of about 8,000 daltons. In at least one embodiment, the block subunits of polyalkylene oxide in the TPU polymer can have a formula weight of about 12.000 daltons. In at least one embodiment, the block subunits of polyalkylene oxide (e.g., polyethylene glycol) in the TPU polymer can have a formula weight of between 1000 and 2000 daltons (inclusive), about 6,000 daltons, a formula weight of about 8,000 daltons, a formula weight of about 12,000 daltons, a formula weight of about 6,000 daltons, or a mixture of block subunits having any two or more of the foregoing formula weights. It is understood that these molecular weight values are average values and refer to the weight average molecular weight.

In addition, ionic groups can be added into the polymer backbone by addition of compounds that have ionic groups that are capable of reacting into the polymer. Examples include sulfopolyester polyols derived from Sulfoarylene- and sulfoalkylenedicarboxylic acids that may be useful for preparation of the sulfocompounds of the invention are any of the known sulfoarene- and sulfoalkanedicarboxylic acids. Examples of these include sulfoalkanedicarboxylic acids such as sulfosuccinic acid, 2-sulfoglutaric acid, 3-sulfoglutaric acid and 2-sulfododecanedioic acid, sulfoarenedicarboxylic acids such as 2-sulfoterephthalic acid, 5-sulfonaphthalene-1,4-dicarboxylic acid, and 5-sulfoisophthalic acid, which is preferred; sulfobenzylmalonic acids such as those described in U.S. Pat. No. 3,821,281; and sulfofluorene-dicarboxylic acids such as 9,9-di(2′-carboxyethyl)fluorene-2-sulfonic acid described in British Patent No. 1,006,579; all references are incorporated herein by reference. It is to be understood that the corresponding lower alkyl esters, halides, anhydrides, and salts of the above sulfonic acids can also be used in the preparation. Also useful are carboxylic acid functional compounds such as dimethylolpropionic acid (DMPA) which can be reacted e.g. with polyisocyanates to form polyurethanes.

The second component can also have a proportion of groups derived from aromatic or aliphatic polyisocyanates. For example, the polyalkylene oxide segments are reacted with diisocyanates to form a relatively high MW extrudable polyurethane. Typical polyisocyanates include the following: 2,4-toluene diisocyanate, 2,6-toluene diisocyanate, mixtures of these isomers, 4,4′-diphenylmethane diisocyanate, 2,4′-diphenylmethane diisocyanate, mixtures of these isomers together with possible small quantities of 2,2′-diphenylmethane diisocyanate (typical of commercially available diphenylmethane diisocyanate), and aromatic polyisocyanates and their mixtures such as are derived from phosgenation of the condensation product of aniline and formaldehyde, tetramethylene diisocyanate, hexamethylenediisocyanate (HDI), dodecamethylenediisocyanate, 1,4-diisocyanatocy-clohexane, 1-isocyanato-3,3,5-trimethyl-5-isocyanatomethylcyclohexane (IPDI), 4,4′-diisocyanato-dicyclohexylmethane (H12 MDI), 4,4′-diisocyanato-2,2-dicyclohexyl-propane, 1,4-diisocyanatobenzene, 2,4-diisocyanatotoluene (TDI), 2,6-diisocyanatotoluene, 4,4′-diisocyanatodiphenylmethane (MDI), m- and p-xylylenediisocyanate, .alpha.,.alpha.,.alpha.′-,.alpha.′-tetramethyl-m- and p-xylylenediisocyanate and mixtures of these compounds. Especially preferred diisocyanates include IPDI, H12 MDI, HDI, TDI and MDI. Most preferred diisocyanates include IPDI and H12 MDI. For example, the second component can also include from 10 to 25 wt. % 4,4′-diisocyanato-dicyclohexylmethane (H₁₂MDI).

Other optional materials can be added to the compositions and constructions (e.g., as additives and/or coatings) used in the present invention to impart desirable properties such as handling, processability, stability, and dispersability to the resulting articles. Nonlimiting examples of other materials include plasticizers, antimicrobial agents, fluid repellents, surfactants, dispersing agents, antioxidants, fillers, nucleants, crosslinkers as well as antistatic, foaming agents, colorants, pharmaceutical compositions, waxes, and talcs.

Nonlimiting examples of plasticizers include triethyl citrate, alkyl lactates, triacetin, alkyl glycols, and oligomers of the base polymer and can be present in amounts ranging from about 1 to about 50 weight percent of the final composition and preferably in an amount ranging from about 5 to about 30 weight percent. Plasticizers useful can include, but are not limited to, polyethylene glycol; polyethylene oxide; citrate esters (such as tributyl citrate oligomers, triethyl citrate, acetyltributyl citrate, acetyltriethyl citrate); glucose monoesters; partially fatty acid esters; PEG monolaurate; triacetin; poly([epsilon]-caprolactone); poly(hydroxybutyrate); glycerin-1-benzoate-2,3-dilaurate; glycerin-2-benzoate-1,3-dilaurate; starch; bis(butyl diethylene glycol)adipate; glycerine diacetate monocaprylate; diacetyl monoacyl glycerol polypropylene glycol (and epoxy, derivatives thereof); polypropylene glycol)dibenzoate, dipropylene glycol dibenzoate; glycerol; ethyl phthalyl ethyl glycolate; poly(ethylene adipate)distearate; di-iso-butyl adipate; diethyl phthalate, p-toluene ethyl sulfonamide, triphenyl phosphate, triethyl tricarballylate, methyl phthallyl ethyl glycolate, sucrose octaacetate, sorbitol hexaacetate, mannitol hexaacetate, pentaerythritol tetraacetate, triethylene diacetate, diethylene dipropionate, diethylene diacetate, tributyrin, tripropionin, and combinations thereof. In some embodiments, the plasticizer is selected based on its compatibility with the first and second materials and based on the conditions under which the multicomponent filament will be used.

Antimicrobial agents are known to those skilled in the art. While it is not presently known which specific antimicrobial agents, antifungal agents, and the like would be compatible in these constructions and compositions of the present invention, nonlimiting examples might include cationic compounds such as copper and silver compounds, benzalkonium chloride, cetyltrimethylammonium halides, polyhexamethylene biguanide, chlorhexidine salts such as acetate, lactate, and glucanate, iodophores, pyrithiones, isothiazolines, or benzimidazoles. A nonvolatile carrier also can be added to improve the antimicrobial activity. Particularly preferred are those antimicrobials and antimicrobial carriers disclosed in US Patent Application Publication No. 20080200890 incorporated herein by reference. These agents may be present in amounts ranging from about 0.05% by weight to 5% by weight depending on the agent and based on the total composition.

Surfactants can be used to improve the hydrophilicity of the filaments. Useful surfactants (also known as emulsifiers) can be either coated onto the multicomponent filament or incorporated into the polymer melt. Preferred surfactants are anionic, zwitterionic, and nonionic. Surfactants include anionic surfactants, such as alkylarylether sulfates and sulfonates such as sodium alkylarylether sulfate (e.g., sulfonated nonylphenol ethoxylates such as those known under the trade designation “TRITON X200”, available from Rohm and Haas, Philadelphia. Pa.), alkylarylpolyether sulfates and sulfonates (e.g., alkylarylpoly(ethylene oxide) sulfates and sulfonates, preferably those having up to about 4 ethyleneoxy repeat units), and alkyl sulfates and sulfonates such as sodium lauryl sulfate, ammonium lauryl sulfate, triethanolamine lauryl sulfate, and sodium hexadecyl sulfate, alkyl ether sulfates and sulfonates (e.g., ammonium lauryl ether sulfate, and alkylpolyether sulfate and sulfonates (e.g., alkyl poly(ethylene oxide) sulfates and sulfonates, preferably those having up to about 4 ethyleneoxy units). Alkyl sulfates, alkyl ether sulfates, and alkylarylether sulfates are also suitable. Additional anionic surfactants can include alkylaryl sulfates and sulfonates (e.g., sodium dodecylbenzene sulfate and sodium dodecylbenzene sulfonate), sodium and ammonium salts of alkyl sulfates (e.g., sodium lauryl sulfate, and ammonium lauryl sulfate); nonionic surfactants (e.g., ethoxylated oleoyl alcohol and polyoxyethylene octylphenyl ether); and cationic surfactants (e.g., a mixture of alkyl dimethylbenzyl ammonium chlorides, wherein the alkyl chain contains from 10 to 18 carbon atoms). Zwitterionic surfactants are also useful, and include sulfobetaines, N-alkylaminopropionic acids, and N-alkylbetaines. A nonvolatile carrier also can be added to improve the wetting and absorbency. Particularly preferred surfactants and carriers are disclosed in U.S. Pat. No. 8,858,986 incorporated herein by reference.

An optional additive can also comprise a secondary crosslinker that crosslinks the first and/or the second component. Crosslinking the first and/or second component can result in higher wet tensile strength. Secondary crosslinkers can comprise peroxides, or polyisocyanates.

The secondary crosslinker can be added with either the first component or second component. However, a secondary crosslinker is not required for the crosslinking to occur as discussed herein.

The multicomponent filament can have hydrophilic characteristics that produce a balance of slip and grip properties. One measure of the hydrophilic characteristics of the multicomponent filament is the absorption capacity of the filament. The selection of the second component can be such that the resulting absorption of the multicomponent fiber is no greater than 9 grams, no greater than 8 grams, no greater than 7 grams, no greater than 6 grams, no greater than 5 grams, or no greater than 4 grams deionized water at 25 degrees Celsius per gram of multicomponent filament as determined by the lubricity test method described herein.

The absorption can be affected by the amount of second component within the multicomponent filament relative to the first component. For example, a multicomponent filament with a higher proportion of second component can have greater absorption capacity than a multicomponent filament with a lower proportion of second component. The absorption capacity of the second component can be 200-1500 wt. % deionized water at 25 degrees Celsius per weight of the second component based on the absorption test method described herein.

The coefficient of friction can be highly variable and be difficult to measure for a single fiber. Therefore, the coefficient of friction can be measured based on articles formed from the multicomponent filament or yarn as demonstrated in the lubricity test method described herein. Particularly, it was found that knitted articles having a balance of slip and grip properties described herein have a coefficient of friction that is 0.2-0.5 using the lubricity test method.

The multicomponent filament can be assembled by combining the first component and the second component in a variety of possible configurations. Suitable multicomponent filament configurations include, but are not limited to, a sheath-core configuration, segmented ribbon, segmented cross, tipped trilobal, half moon, hollow pie wedge, conjugate, a side-by-side, a layered or a segmented pie/wedge configuration (for example, U.S. Pat. No. 4,729,371 describes layered bi-component meltblown fibers, also referred to as striped fibers; and PCT International Publication No. WO 2008/085545 describes segmented pie/wedge fibers and layered fibers), and an “islands-in-the-sea” configuration (for example, fibers produced by Kuraray Company, Ltd., Okayama, Japan).

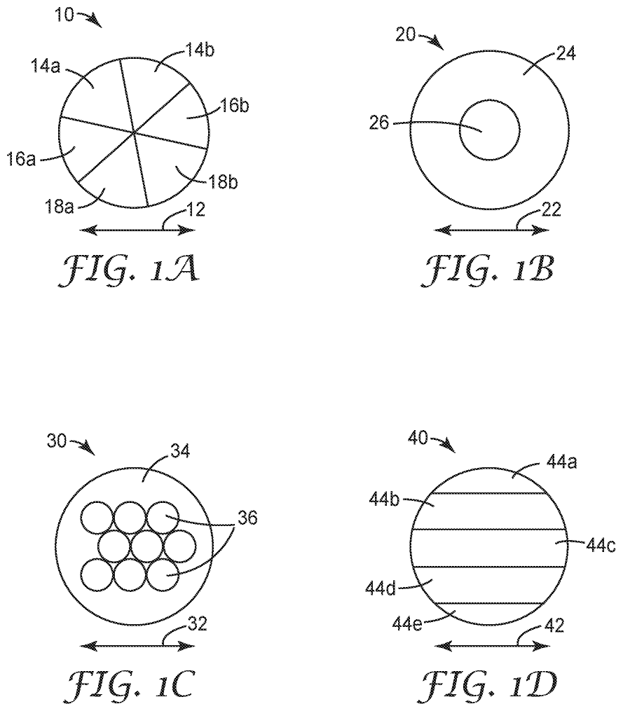

Referring to FIG. 1A, pie-wedge filament 10 has a circular cross-section 12, and first component 14a and 14b, second component 16a and 16b, and optional components 18a and 18b.

In FIG. 1B, multicomponent filament 20 has circular cross-section 22 and second component sheath 24, and first component core 26. This figure shows the sheath completely enveloping the core. In some embodiments, the sheath may extend around at least 75, 80, 85, 90, 95, 97, or 99 percent of the outer surface of core or cores. The sheath can comprise at least 10, at least 20, at least 30, at least 40, at least 50, at least 60, at least 70, at least 80, or at least 90 (dry) weight percent of the resulting multicomponent filament.

FIG. 1C shows multicomponent filament 30 that has a circular cross-section 32. The multicomponent filament 30 has an islands in the sea configuration with a second component sea 34 and plurality of first component islands 36. An islands-in-the sea configuration can be made according to the example shown in U.S. Pat. No. 4,239,720.

FIG. 1D shows multicomponent filament 40 having circular cross-section 42, with five layered regions 44a, 44b, 44c, 44d, 44e, which comprise alternatively at least the first and second components described herein, e.g., 44a, 44c and 44e are the second component and 44b and 44d are the first component.

The absorptive properties of the second component may be arranged to provide a greater absorptive effect. For example, in a medical application, a sheath-core or islands-in-the-sea configuration may provide a larger hydrophilic surface area relative to a side-by-side, pie-wedge, or layered configuration to absorb liquids.

Filaments described herein can generally be made using techniques known in the art for making filaments. Such techniques include wet spinning, dry spinning, melt spinning, or gel spinning.

Particularly advantageous to form the multicomponent filament is melt spinning. In melt spinning, a polymer is heated, passed through a spinneret, and fibers solidify upon cooling. For example, a melt spinning process can occur to collect the multicomponent filaments. The term “meltspun” as used herein refers to filaments that are formed by extruding molten filaments out of a set of orifices and allowing the filaments to cool and (at least partially) solidify to form filaments, with the filaments passing through an air space (which may contain streams of moving air) to assist in cooling and solidifying the filaments, and with the thus-formed fibers then passing through an attenuation (i.e., drawing) unit to draw the fibers. Meltspinning can be distinguished from meltblowing, which involves the extrusion of molten filaments into converging high velocity air streams introduced by way of air-blowing orifices located in close proximity to the extrusion orifices. Meltspinning can also be distinguished from electrospinning in that electrospinning could be described as extruding out of a need a solvent solution.

A modification of the spinneret results in multicomponent (e.g., bi-component) fibers. (See, e.g., U.S. Pat. No. 4,406,850 (Hills), U.S. Pat. No. 5,458,972 (Hagen), U.S. Pat. No. 5,411,693 (Wust), U.S. Pat. No. 5,618,479 (Lijten), and U.S. Pat. No. 5,989,004 (Cook)). Filaments according to the present disclosure can also be made by fibrillation of a film, which may provide filaments having a rectangular cross-section.

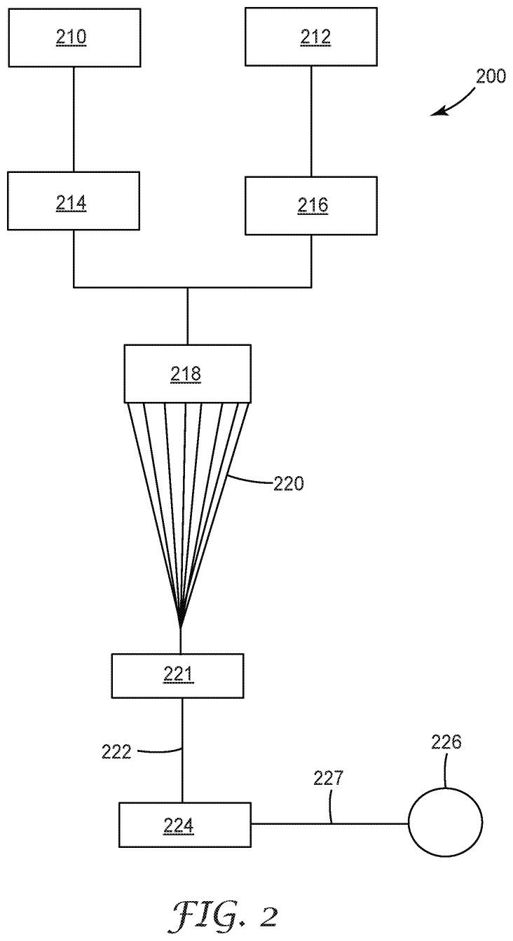

FIG. 2 illustrates a system 200 for making the multicomponent yarn of the present disclosure. Although the system 200 is shown with two separate extruders (extruder 214 and extruder 216), the system 200 is contemplated with any number of extruders including one extruder that coextrudes both the first and second component. Various extruders can be used. For example, a 25 mm twin screw extruder (commercially available under the trade designation “Ultraglide” from Berstorff, Hannover, Germany) can be used to extrude the first component and/or the second component.

Each source can contain a component. For example, source 210 can contain the first component while source 212 can contain the second component. An extruder can melt the polymer that is fed from source 210 and 212. The extruder can apply temperature and pressure to each source to enhance processing by an extruder. The multicomponent filament can be made by (co)extruding (via at least one of the extruders 214, 216) a first component (from source 210) and a second component (from source 212) through a spinneret 218 to form at least one pre-multicomponent filament 220.

At least one extruder can be heated to various temperatures. The temperature of the at least one extruder will vary depending on the type of materials selected for use as the first and second component. The first and second component can be heated to different temperatures sufficient for a first viscosity of the first component to approach the second viscosity of the second component. The first source 210 can be introduced into the spinneret 218 at a first melt temperature and the second source 212 may be introduced into the spinneret 218 at a second melt temperature. The difference between the first melt temperature and the second melt temperature can be at least 5° C., 10° C., 20° C., 30° C., at least 40° C., at least 60° C., at least 80° C. or at least 100° C. depending on the first and/or second component used.

The melt temperature of the extruder may also be varied. For example, a first component comprising an ether-based TPU can have a first melt temperature that ranges from 10° C. to 200° C. (inclusive), including 80° C. to 120° C. (inclusive), 95° C. to 120° C. (inclusive), or 150° C. to 180° C. (inclusive) or 100° C. to 136° C. (inclusive).

The die temperature for the extruder refers to a spinneret die 218 that combines the first source 210 and second source 212. The die temperature may be similar for the first component and the second component. Depending on the component selection, the die temperature can range from 60° C.-500° C. Exemplary die temperatures can be at least 100° C., at least 180° C., at least 190° C., at least 200° C., at least 210° C., at least 220° C., at least 230° C., at least 240° C., at least 250° C., or at least 300° C. In at least one embodiment. The die temperature for the extruder can be no greater than 350° C., no greater than 300° C., no greater than 290° C., no greater than 280° C., or no greater than 270° C., or any combination with the aforementioned die temperature.

A plurality of filaments 220 can be drawn and processed into a multicomponent yarn 222. For example, once extruded, the resulting filaments are drawn by attenuating unit 221 to form a multicomponent filament 220. During the processing, the multicomponent filament 220 are grouped or arranged into a multicomponent yarn. Cooling can be done under ambient conditions using air or by using any known cooling techniques.

The diameter of the multicomponent filament 220 will depend on a number of factors, such as ratio of first and second components, configuration of a multicomponent filament, processing conditions, and the degree of absorption or flexural strength required by an application of a multicomponent filament. A higher diameter multicomponent filament in a sheath-core configuration with a high proportion of second component to first component will likely have a higher absorptivity. Exemplary diameters of the multicomponent filament are no greater than 100 micrometers, no greater than 50 micrometers, no greater than 35 micrometers, no greater than 34 micrometers, no greater than 33 micrometers, no greater than 32 micrometers, no greater than 31 micrometers, no greater than 30 micrometers, or no greater than 29 micrometers.

A plurality of multicomponent filaments 220 can be optionally grouped together to form a multicomponent yarn 222. The diameter of the multicomponent filament 220 can be influenced by drawing of the filament when collected as a multicomponent yarn 222. For example, a high tension (i.e., high draw) can reduce the diameter of a filament 220.

A multicomponent yarn 222 or multicomponent filament 220 can be crosslinked by, for example, a radiation source 224. Prior to crosslinking, the multicomponent yarn 222 can be thermoplastic and have free hydrophilic groups.

Crosslinking may enhance the wet tensile strength of the multicomponent fiber and decrease the friction. Crosslinking may reduce the water solubility of hydrophilic groups from the multicomponent filament 220 by linking them with other reactive elements in the multicomponent filament 220. While the crosslinking can improve the wet durability of the multicomponent filament 220 as well as the adhesion or cohesion between the first and second components, the crosslinking may also reduce the absorption capacity of the multicomponent filament 220. In at least one embodiment, the radiation source 224 can be ultraviolet, electromagnetic, proton beam, neutron beam, or electron beam.

Ultraviolet (UV) radiation can occur at an exposure sufficient to crosslink the multicomponent filament. For example, the multicomponent filament 220 can be exposed to 0-500 mJ/cm² UVB. Various initiators may be added to the polymer of the first and/or second component of the multicomponent filament 220 to facilitate the crosslinking.

Electron beam radiation can be applied at an exposure to sufficiently crosslink the multicomponent filament 220 to the desired absorption or lubricity. The electron beam can be applied at a dosage of at least 1 Mrad, at least 3 Mrads, at least 5 Mrads, at least 10 Mrads, at least 15 Mrads, at least 20 Mrads and no greater than 25 Mrads.

After crosslinking, the multicomponent filament 220 is referred to as a crosslinked multicomponent filament and the multicomponent yarn 222 is referred to as a crosslinked multicomponent yarn 227. Throughout this disclosure, the term multicomponent yarn can be used to refer to a crosslinked multicomponent yarn or a thermoplastic multicomponent yarn.

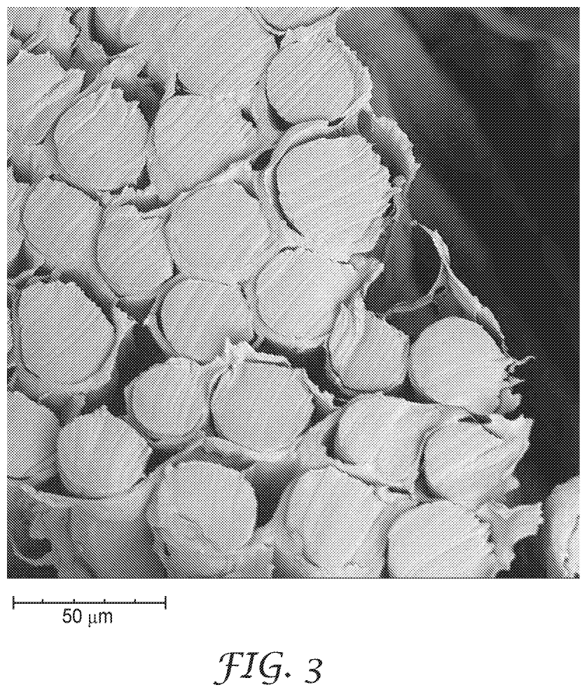

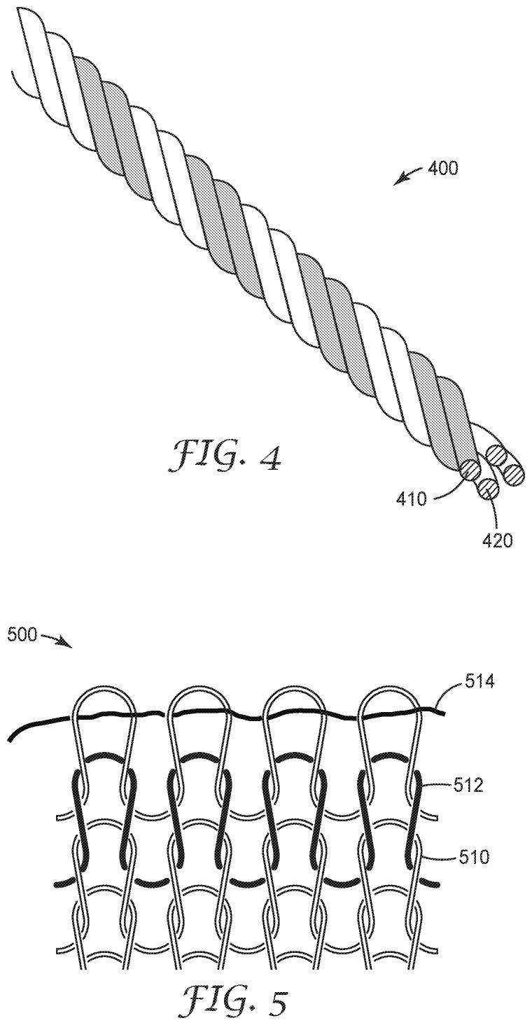

Once crosslinked, the crosslinked multicomponent yarn 227 (e.g., FIG. 3 shows an untwisted yarn in example 2 which is described herein) can be drawn onto a spool 226 (in addition to the drawing of filaments at the spinerette 218). Additional post-drawing can be performed which can stretch the filaments to an desired diameter. Drawing can be done at various roll speeds depending on the selection of first and second components and the desired resulting diameter of the controlled degradation fibers. For example, a roll speed of at least 50 m/min, at least 100 m/min, at least 150 m/min, at least 200 m/min, at least 250 m/min, at least 350 m/min, at least 550 m/min, at least 650 m/min, or at least 750 m/min can be used. Fiber melt spinning speed can go up to 8000 m/min in traditional manufacturing equipment. The demonstrated line speeds in the examples were performed on a smaller scale and was limited by the partial function winding system used.

An aspect of the present disclosure is that a multicomponent filament 220 is manufactured using a solvent-free process meaning that neither the first component nor the second component are dissolved in solvent prior to extrusion. The first component and the second component can be melt processable meaning that the first component and the second component are melted and subjected to extrusion and temperature in a continuous process.

Articles of the present disclosure can include a first yarn and optional second yarn. The first yarn comprises at least one hydrophilic filament. The hydrophilic filament contributes to partial and/or the overall absorption of the knitted article. Hydrophilic filaments can be formed from a single component made from a hydrophilic material such as the second component described herein. In at least one embodiment, the hydrophilic filament includes the multicomponent filament discussed herein. Thus, the first yarn can include the multicomponent filament. In at least one embodiment, the first yarn can be comprised of at least 5 wt. %, 10 wt. %, 20 wt. %, 30 wt. %, 40 wt. %, 50 wt. %, at least 60 wt. %, at least 70 wt. %, at least 80 wt. %, at least 90 wt. % of the hydrophilic filament (e.g., the multicomponent filament).

The secondary fiber can enhance grip of a yarn. The secondary fiber can also add structural strength to a yarn. In at least one embodiment, a second yarn includes a secondary fiber. In another embodiment, the first yarn can be spun with the secondary fiber.

The secondary fiber can include any variety of filaments and fibers. For example, the secondary fiber can include various natural or synthetic components (e.g., rayon, cotton, polyethylene, polypropylene, polyester, polyamide, polyurethane, spandex, silk, wool, viscose or combinations thereof). The secondary fiber can also comprise a radiopaque element that is visible using x-rays. For example, the radiopaque element can be polyvinyl chloride having barium sulfate incorporated.

Various processing techniques can modify the properties of a yarn (e.g., twisting staple fibers or filaments, such as secondary fibers or filaments including cotton, rayon, cellulose, together in S or Z twists). The twisting may enhance grip at the expense of lubricity when the yarn is wetted. Multicomponent yarns used in the present invention can be made, e.g., by melt spinning any number of filaments together to make a sheath-core fiber tow which contain barely any twists.

In FIG. 4, an exemplary first yarn 400 according to one embodiment of the present invention includes a number of individual monofilaments or fibers. The first yarn can comprise at least one multicomponent filament 410 and a secondary fiber 420. The monofilaments and fibers can be combined together by up-twisting the individual monofilaments and a second yarn comprising the secondary fiber together using either an S-twist or Z twist direction with a significant number of 360 degree turns per inch of yarn length.

In FIG. 4, the yarn 400 includes at least two individual monofilaments, each a highly resilient filament having a tex of at least 100. Such a yarn can also be formed using as few as two individual filaments or yarns, for example a 200 tex, two filament yarn, designated as 200/2 (200 tex/2 filaments). The optional secondary fiber 420 can be in the form of a second yarn comprised of several staple fibers.

In the embodiment in FIG. 4, the yarn has a twist of 10 to 16 minimum turns per inch along the length of the yarn. In other embodiments, a yarn according to the present invention can have as few as one or two twists per centimeter or inch of yarn length. The 200 tex, 4 filament yarn as illustrated in FIG. 4 can be air entangled instead of twisted.

The filaments may be air entangled with as few as 2 entanglement nodes per meter of length and as many as up to 8, 10, 12, or 14 entanglement nodes per meter of yarn length. The dynamics of twisting individual fibers or filaments into a composite yarn can produce varied degrees of stiffness and stability depending on the number of turns per inch of twist that are induced. In general, as the number of turns per inch are increased, the stiffness index and resilience, or resistance to bending, characteristics of the yarn increase proportionally.

The first yarn can be made using a variety of spinning techniques. For example, break, mule, or open-end spinning can be used to create a yarn. Both the hydrophilic fiber and the secondary fiber can contribute to the overall wet tensile strength of the first yarn.

A knitted article described herein can be used in a medical application. The knitted article can comprise the first yarn described herein and a second yarn. The second yarn can be made of a variety of materials such as the secondary fiber described herein. For example, if a hydrophilic fiber constitutes a majority of the first yarn, then a second yarn can be made primarily of a secondary fiber. The second yarn can be made by twisting staple fibers to form a spun yarn. The composition of the second yarn can be at least 5, 10, 20, 30, 40, 50, at least 60, at least 70, at least 80, or at least 90 wt. % secondary fiber relative to the second yarn.

The multicomponent yarn described herein can be used to form a variety of articles. The coefficient of friction of the article can have applications beyond surgical and include medical and personal care applications, such as tissue friendly contacting materials, wound dressing, absorbent pad, suture, feminine hygiene, and cosmetic facial masks.

The coefficient of friction of the knitted article can be influenced by a first yarn comprising a hydrophilic filament (such as the multicomponent filament) and an optional secondary fiber. The coefficient of friction of the knitted article can also be influenced by a second yarn comprising the optional secondary fiber. An aspect of the present disclosure is that the coefficient of friction of the knitted article can be tuned by the second yarn (e.g., the secondary fiber).

FIG. 5 illustrates an exemplary knitted article 500 with a stockinette type knitting pattern. The knitted article 500 can have a first yarn 510 and a second yarn 512. The first yarn 510 and second yarn 512 can use materials sufficient to make the resulting knitted article 500 have a coefficient of friction between 0.2 and 0.5. Depending on the composition of the yarns, the knitted article 500 can comprise at least 5 wt. %, 10 wt. %, 20 wt. %, 30 wt. %, 40 wt. %, 50 wt. %, at least 60 wt. %, at least 70 wt. %, at least 80 wt. %, or at least 90 wt. % of first yarn relative to the knitted article.

In some embodiments, the knitted article 500 can be formed from the first yarn 510 and a second yarn 514 can later be threaded through the knitted article 500. As shown, second yarn 512 illustrates a linked thread through and second yarn 514 illustrates an unlinked thread through (and is thus not structural)(also referred to as a chain stitch) in the knitted article 500.

Various knitting patterns include, for example, stockinette stitch, garter stitch, plaited stitch, tuck stitch. Preferably, the knitted article can be knitted using automated machinery. The end product can be used as a fabric for garments, used for cleaning, or medical articles. For example, a knitted bandage or a laparotomy sponge can be created using the knitted article.

Once the knitted article is formed, then the knitted article can be further processed. The knitted article can be further processed with various durability coatings, antibacterial or antifungal solutions, sterilization, and crosslinking (described herein).

Sterilization and crosslinking can occur in the same process. For example, irradiating the knitted article can both crosslink the polymers in the knitted article and sterilize. The irradiating comprises Ultraviolet (UV), electron beam, gamma, or other types of radiation treatment described herein to create a knitted article with the desired coefficient of friction and structure.

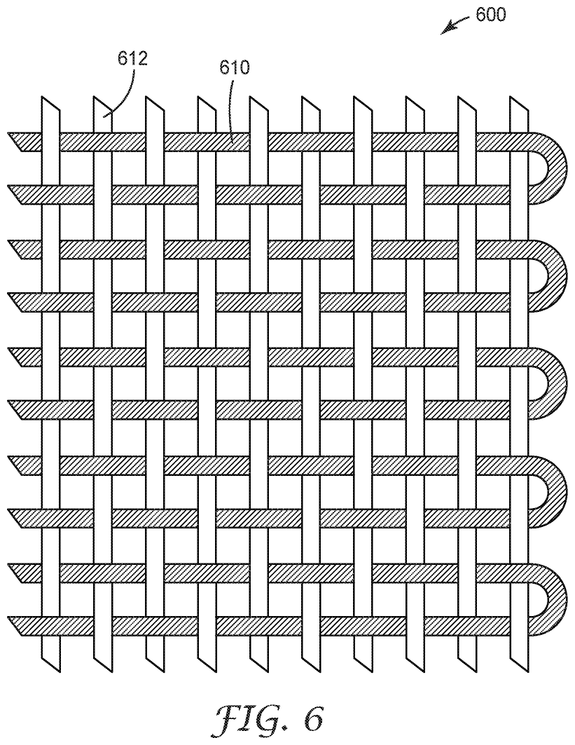

Aspects of the present disclosure can also include a woven article comprised of a first yarn or a hydrophilic fiber. The woven article can also include a secondary fiber to tune the properties of the woven article. FIG. 6 illustrates a woven article 600 prepared using a hydrophilic fiber 610 and an optional secondary fiber 612. The woven article 600 demonstrates a plain weave but a variety of weaves are possible, for example, basket weave, satin weave, twill weave, etc. The weave may result in different lubricity for the woven article 600.

The woven article 600 can be created using a variety of conventional techniques. For example, the woven article can be a woven fabric created using an air jet loom, a water jet loom, and rapier loom.

The woven article 600 can have a blend of the first yarn 610 and the second yarn 612 sufficient to have a coefficient of friction between 0.2 and 0.5 using the lubricity test method.

Although not pictured, aspects of the present disclosure can also include a nonwoven article. The nonwoven article can have at least a portion of a multicomponent fiber. The nonwoven article can be prepared using a variety of methods, for example, meltblowing, wet laid, needle tacking, chain stitching into a nonwoven, carding, etc. Exemplary methods for preparing a nonwoven can be found on U.S. Pat. No. 9,487,893. The nonwoven article can also include a secondary fiber. The secondary fiber may be incorporated into the nonwoven as a meltblown fiber or as a staple fiber. Additionally, the secondary fiber can be added by chain stitching onto a nonwoven article including the multicomponent filament.

### LIST OF ILLUSTRATIVE EMBODIMENTS

### Embodiment 1a

A Multicomponent Filament, Comprising:

a first component comprising a thermoplastic polymer; and

a second component comprising a hydrophilic thermoplastic polymer comprising hydrophilic segments.

### Embodiment 1b

The multicomponent filament of embodiment 1a, wherein the second component comprises 65% (w/w) to 90% (w/w), inclusive, hydrophilic segments.

### Embodiment 1c

The multicomponent filament of any of the preceding embodiments, wherein the first component is capable of forming a continuous filament with the second component.

### Embodiment 1d

The multicomponent filament of any of the preceding embodiments, wherein the hydrophilic segments comprise a polyalkylene oxide.

### Embodiment 1e

The multicomponent filament of any of the preceding embodiments, wherein the hydrophilic segments are selected from the group consisting of polyethylene glycol, polypropylene glycol, polybutylene oxide, random poly(C2-C4)alkylene oxide, polyester, amine-terminated polyester, amine-terminated polyamide, polyester-amide, polycarbonate, and combinations thereof.

### Embodiment 1f

The multicomponent filament of any of the preceding embodiments, wherein the hydrophilic thermoplastic polymer is an aliphatic polyether thermoplastic polyurethane polymer having at least 65% (w/w) polyalkylene oxide.

### Embodiment 1g

The multicomponent filament of embodiment 1d, wherein the polyalkylene oxide is polyethylene glycol.

### Embodiment 1h

The multicomponent filament of embodiment 1g, wherein the polyalkylene glycol is 70-90% by weight.

### Embodiment 1i

The multicomponent filament of embodiment 1h, wherein the polyethylene glycol subunits have a formula weight of at least 1000 daltons.

### Embodiment 2

The multicomponent filament of any of the preceding embodiments, wherein the multicomponent filament is capable of being spun into a multicomponent yarn.

### Embodiment 3

The multicomponent filament of any of the preceding embodiments, wherein the thermoplastic polymer has a melt flow index from 10 g/10 min to 100 g/10 min inclusive at 190° C. (inclusive).

### Embodiment 4

The multicomponent filament of any of the preceding embodiments, wherein the thermoplastic polymer has a melt flow index from 10 g/10 min to 80 g/10 min inclusive at 190° C. (inclusive).

### Embodiment 5

The multicomponent filament of any of the preceding embodiments, wherein the thermoplastic polymer has a melt flow index from 20 g/10 min to 40 g/10 min inclusive at 190° C. (inclusive).

### Embodiment 6a

The multicomponent filament of any of the preceding embodiments, wherein the thermoplastic polymer has a melt flow index from 25 g/10 min to 35 g/10 min inclusive at 190° C. (inclusive).

### Embodiment 6b

The multicomponent filament of any of the preceding embodiments, wherein the thermoplastic polymer has a melt flow index from 26 g/10 min to 34 g/10 min inclusive at 190° C. (inclusive).

### Embodiment 6c

The multicomponent filament of any of the preceding embodiments, wherein the thermoplastic polymer has a melt flow index from 27 g/10 min to 33 g/10 min inclusive at 190° C. (inclusive).

### Embodiment 6d

The multicomponent filament of any of the preceding embodiments, wherein the thermoplastic polymer has a melt flow index from 28 g/10 min to 32 g/10 min inclusive at 190° C. (inclusive).

### Embodiment 6e

The multicomponent filament of any of the preceding embodiments, wherein the thermoplastic polymer has a melt flow index from 29 g/10 min to 31 g/10 min inclusive at 190° C. (inclusive).

### Embodiment 7

The multicomponent filament of any of the preceding embodiments, wherein the thermoplastic polymer has a melt flow index of about 30 g/10 min inclusive at 190° C. (inclusive).

### Embodiment 8a

The multicomponent filament of any of the preceding embodiments, wherein the thermoplastic polymer has a melting temperature from 90 degrees C. to 250 degrees C. (inclusive).

### Embodiment 8b

The multicomponent filament of any of the preceding embodiments, wherein the thermoplastic polymer has a melting temperature from 90 degrees C. to 190 degrees C. (inclusive).

### Embodiment 8c

The multicomponent filament of any of the preceding embodiments, wherein the thermoplastic polymer has vicat softening temperature of 40 degrees C. to 100 degrees C. using ASTM D2240.

### Embodiment 9

The multicomponent filament of any of the preceding embodiments, wherein the second component includes few enough hydrophilic functional groups such that the multicomponent filament exhibits water absorption no greater than 9 grams water per gram multicomponent filament according to the yarn absorption test method.

### Embodiment 10

The multicomponent filament of any of the preceding embodiments, wherein the thermoplastic polymer is selected from a group consisting of: styrenic block copolymers, thermoplastic olefins, elastomeric alloys, acrylic block copolymers, thermoplastic polyurethanes, thermoplastic copolyesters, and thermoplastic polyamides.

### Embodiment 11

The multicomponent filament of any of the preceding embodiments, wherein the thermoplastic polyurethanes comprises a polyester-based thermoplastic polyurethane.

### Embodiment 12

The multicomponent filament of any of the preceding embodiments, wherein the thermoplastic polymer comprises a thermoplastic olefin.

### Embodiment 13

The multicomponent filament of any of the preceding embodiments, wherein the thermoplastic olefin comprises a polyethylene.

### Embodiment 14

The multicomponent filament of any of the preceding embodiments, wherein the polyethylene comprises a linear low density polyethylene.

### Embodiment 15

The multicomponent filament of any of the preceding embodiments, wherein the thermoplastic olefin comprises a polypropylene.

### Embodiment 16

The multicomponent filament of any of the preceding embodiments, wherein the thermoplastic olefin comprises a polymethylpentane.

### Embodiment 17

The multicomponent filament of any of the preceding embodiments, wherein the thermoplastic olefin comprises a polybutene-1.

### Embodiment 18

The multicomponent filament of any of the preceding embodiments, wherein the thermoplastic copolyester comprises a thermoplastic aliphatic polyester.

### Embodiment 19

The multicomponent filament of any of the preceding embodiments, wherein the thermoplastic aliphatic polyester comprises polylactic acid.

### Embodiment 20

The multicomponent filament of any of the preceding embodiments, wherein the thermoplastic polyurethane of the first component comprises a polyether-based thermoplastic polyurethane.

### Embodiment 21

The multicomponent filament of any of the preceding embodiments, wherein the polyether-based thermoplastic polyurethane of the second component comprises at least 70% (w/w) polyalkylene oxide.

### Embodiment 22

The multicomponent filament of any of the preceding embodiments, wherein the polyether-based thermoplastic polyurethane of the second component comprises at least 72% (w/w) polyalkylene oxide.

### Embodiment 23

The multicomponent filament of any of the preceding embodiments, wherein the polyether-based thermoplastic polyurethane of the second component comprises no greater than 99% (w/w) polyalkylene oxide.

### Embodiment 24

The multicomponent filament of any of the preceding embodiments, wherein the polyether-based thermoplastic polyurethane of the second component comprises no greater than 90% (w/w) polyalkylene oxide.

### Embodiment 25

The multicomponent filament of any of the preceding embodiments, wherein the polyether-based thermoplastic polyurethane of the second component comprises no greater than 85% (w/w) polyalkylene oxide.

### Embodiment 26

The multicomponent filament of any of the preceding embodiments, wherein the polyether-based thermoplastic polyurethane of the second component comprises no greater than 80% (w/w) polyalkylene oxide.

### Embodiment 27

The multicomponent filament of any of the preceding embodiments, wherein the first component is an island and second component is a sea in an islands-in-the-sea multicomponent filament.

### Embodiment 28

The multicomponent filament of any of the preceding embodiments, wherein the first component is the core and second component is the sheath in a core/sheath multicomponent filament.

### Embodiment 29

The multicomponent of any of the preceding embodiments, wherein the second component comprises at least one of the group consisting of: antioxidants, antistatic, foaming agents, pharmaceutical compositions, plasticizers, antimicrobial agents, fluid repellents, or combinations thereof.

### Embodiment 30

The multicomponent filament of any of the preceding embodiments, wherein the multicomponent filament has an average diameter of no greater than 100 micrometers.

### Embodiment 31

The multicomponent filament of any of the preceding embodiments, wherein the multicomponent filament has an average diameter of no greater than 50 micrometers.

### Embodiment 32

The multicomponent filament of any of the preceding embodiments, wherein the multicomponent filament has an average diameter of no greater than 40 micrometers.

### Embodiment 33

The multicomponent filament of any of the preceding embodiments, wherein the multicomponent filament has an average diameter of no greater than 35 micrometers.

### Embodiment 34

The multicomponent filament of any of the preceding embodiments, wherein the multicomponent filament has an average diameter of no greater than 30 micrometers.

### Embodiment 35

The multicomponent filament of any of the preceding embodiments, wherein the second component is crosslinked.

### Embodiment 36

The multicomponent filament of any of the preceding embodiments, wherein the multicomponent filament does not comprise a secondary crosslinker.

### Embodiment 37

The multicomponent filament of any of the preceding embodiments, wherein the multicomponent filament comprises at least 5% (w/w) of the second component.

### Embodiment 38

The multicomponent filament of any of the preceding embodiments, wherein the multicomponent filament comprises at least 20% (w/w) of the second component.

### Embodiment 39

The multicomponent filament of any of the preceding embodiments, wherein the multicomponent filament comprises at least 25% (w/w) of the second component.

### Embodiment 40

The multicomponent filament of any of the preceding embodiments, wherein the multicomponent filament comprises at least 30% (w/w) of the second component.

### Embodiment 41

The multicomponent filament of any of the preceding embodiments, wherein the multicomponent filament comprises at least 40% (w/w) of the second component.

### Embodiment 42

The multicomponent filament of any of the preceding embodiments, wherein the multicomponent filament comprises at least 50% (w/w) of the second component.

### Embodiment 43

The multicomponent filament of any of the preceding embodiments, wherein the multicomponent filament comprises no greater than 99% (w/w) of the second component.

### Embodiment 44

The multicomponent filament of any of the preceding embodiments, wherein the multicomponent filament comprises no greater than 90% (w/w) of the second component.

### Embodiment 45

The multicomponent filament of any of the preceding embodiments, wherein the multicomponent filament comprises no greater than 80% (w/w) of the second component.

### Embodiment 46

The multicomponent filament of any of the preceding embodiments, wherein the multicomponent filament comprises no greater than 75% (w/w) of the second component.

### Embodiment 47

The multicomponent filament of any of the preceding embodiments, wherein the multicomponent filament comprises no greater than 70% (w/w) of the second component.

### Embodiment 48

The multicomponent filament of any of the preceding embodiments, wherein the multicomponent filament comprises no greater than 60% (w/w) of the second component.

### Embodiment 48a

A first yarn comprising at least the multicomponent filament of any of the preceding embodiments.

### Embodiment 48b

The first yarn of any of the preceding embodiments, wherein the second component includes few enough hydrophilic functional groups such that the coefficient of friction of the first yarn is at least 0.2 and no greater than 0.5 according to a lubricity test method.

### Embodiment 49

The first yarn of any of the preceding embodiments, further comprising a secondary fiber.

### Embodiment 50

The first yarn of any of the preceding embodiments, further comprising a radiopaque element.

### Embodiment 51

The first yarn of any of the preceding embodiments, wherein the radiopaque element comprises barium sulfate.

### Embodiment 52

The first yarn of any of the preceding embodiments, wherein the secondary fiber comprises a natural fiber.

### Embodiment 53

The first yarn of any of the preceding embodiments, wherein the secondary fiber is selected from the group consisting of: rayon, acrylic, cotton, polyethylene, polypropylene, polyester, nylon, viscose, and combinations thereof.

### Embodiment 54

The first yarn of any of the preceding embodiments, wherein the secondary fiber comprises the first component.

### Embodiment 55

The first yarn of any of the preceding embodiments, wherein the secondary fiber is present such that the coefficient of friction of the first yarn is at least 0.2 and no greater than 0.5 according to the lubricity test method.

### Embodiment 56a

An article comprising the first yarn of any of the preceding embodiments.

### Embodiment 56b

The article of any of the preceding embodiments, wherein the article is selected from a group consisting of a nonwoven article, a woven article, a knitted article, and combinations thereof.

### Embodiment 56c

The article of any of the preceding embodiments, wherein the article is a nonwoven article.

### Embodiment 56d

The article of any of the preceding embodiments, wherein the article is a woven article.

### Embodiment 56e

The article of any of the preceding embodiments, wherein the article is a knitted article.

### Embodiment 57

The knitted or woven article of any of the preceding embodiments, further comprising a second yarn comprising the secondary fiber of any of the preceding embodiments.

### Embodiment 58

The knitted or woven article of any of the preceding embodiments, wherein the secondary fiber is present such that the coefficient of friction of the knitted article is at least 0.2 and no greater than 0.5 according to the lubricity test method.

### Embodiment 59

The knitted or woven article of any of the preceding embodiments, wherein the article comprises no greater than 95% (w/w) of the secondary fiber.

### Embodiment 60

The knitted or woven article of any of the preceding embodiments, wherein the article comprises at least 14% (w/w) of the secondary fiber.

### Embodiment 61a

The knitted or woven article of any of the preceding embodiments, wherein the secondary fiber is selected from the group consisting of: rayon, acrylic, cotton, polyethylene, polypropylene, polyester, polyamide, polyurethane, spandex, silk, wool, viscose, and combinations thereof.

### Embodiment 61b

A medical article having the article of any of the preceding embodiments disposed thereon.

### Embodiment 61c

A kit comprising the article of any of the preceding embodiments.

### Embodiment 61d

A kit comprising the medical article of embodiment 61b.

### Embodiment 62

A nonwoven article, comprising at least one filament that further comprises the second component of any of the preceding embodiments.

### Embodiment 63

A nonwoven article, comprising the multicomponent filament of any of the preceding embodiments.

### Embodiment 64

The nonwoven article of any of the preceding embodiments, comprising the secondary fiber of any of the preceding embodiments.

### Embodiment 65

The nonwoven article of any of the preceding embodiments, wherein the secondary fiber is present such that the coefficient of friction of the woven article is at least 0.2 and no greater than 0.5 according to the lubricity test method.

### Embodiment 66

A method of making the multicomponent filament of any of the preceding embodiments, comprising:

extruding molten filaments, through a die, the first component at a first temperature, and the second component at a second temperature, to form a multicomponent filament

### Embodiment 66a

A method of making the multicomponent filament of any of the preceding embodiments, comprising:

meltspinning, through a die, the first component at a first temperature, and the second component at a second temperature, to form a multicomponent filament

### Embodiment 67

The method of embodiment 66, further comprising crosslinking the multicomponent filament to form a multicomponent filament.

### Embodiment 68

The method of any of the preceding embodiments, wherein the crosslinking occurs such that the multicomponent filament exhibits water absorption no greater than 9 grams water per gram multicomponent filament according to the absorption test method.

### Embodiment 69

The method of any of the preceding embodiments, wherein the crosslinking occurs such that the multicomponent filament exhibits water absorption no greater than 6 grams water per gram multicomponent filament according to the absorption test method.

### Embodiment 70

The method of any of the preceding embodiments, wherein the crosslinking occurs such that the multicomponent filament exhibits water absorption no greater than 5 grams water per gram multicomponent filament according to the absorption test method.

### Embodiment 71

The method of any of the preceding embodiments, wherein the crosslinking occurs such that the multicomponent filament exhibits water absorption no greater than 4 grams water per gram multicomponent filament according to the absorption test method.

### Embodiment 72

The method of any of the preceding embodiments, wherein a difference between the first temperature and the second temperature is at least 5° C.

### Embodiment 73

The method of any of the preceding embodiments, wherein a difference between the first temperature and the second temperature is at least 10° C.

### Embodiment 74

The method of any of the preceding embodiments, wherein a difference between the first temperature and the second temperature is at least 25° C.

### Embodiment 75

The method of any of the preceding embodiments, wherein the first temperature is sufficient to cause the first component to have a first viscosity and the second temperature is sufficient to cause the second component to have a second viscosity substantially equal to the first viscosity.

### Embodiment 76

The method of any of the preceding embodiments, wherein crosslinking the second component comprises applying radiation to the multicomponent filament.

### Embodiment 77

The method of any of the preceding embodiments, further comprising:

allowing the multicomponent filament to solidify.

### Embodiment 78

The method of any of the preceding embodiments, further comprising:

drawing the multicomponent filament.

### Embodiment 79

The method of any of the preceding embodiments, further comprising:

grouping a plurality of the multicomponent filaments into a multicomponent yarn.

### Embodiment 80

The method of any of the preceding embodiments, wherein grouping the plurality of multicomponent filaments comprises blending a secondary fiber into the plurality of multicomponent filaments to form the multicomponent yarn such that the resulting yarn has a coefficient of friction of at least 0.2 and no greater than 0.5 according to the lubricity test method.

### Embodiment 81

The method of any of the preceding embodiments, further comprising:

crosslinking the multicomponent yarn.

### Embodiment 82

The method of any of the preceding embodiments, wherein applying radiation comprises electron beaming at a dosage of at least 1 Mrad.

### Embodiment 83

The method of any of the preceding embodiments, wherein applying radiation comprises electron beaming at a dosage of at least 5 Mrads.

### Embodiment 84

The method of any of the preceding embodiments, wherein applying radiation comprises electron beaming at a dosage of at least 10 Mrads.

### Embodiment 85

The method of any of the preceding embodiments, wherein applying radiation comprises electron beaming at a dosage of at least 15 Mrads.

### Embodiment 86

The method of any of the preceding embodiments, wherein applying radiation comprises electron beaming at a dosage of at least 20 Mrads.

### Embodiment 87

The method of any of the preceding embodiments, wherein applying radiation comprises electron beaming at a dosage of at least 25 Mrads.

### Embodiment 88

The method of any of the preceding embodiments, wherein applying radiation comprises using UV light at a dosage of no greater than 500 mJ/cm² UVB.

### Embodiment 89a

The method of any of the preceding embodiments, further comprising:

blending a second yarn of any of the preceding embodiments with the multicomponent yarn such that the resulting yarn has a coefficient of friction of at least 0.2 and no greater than 0.5 according to the lubricity test method.

### Embodiment 89b

The method of any of the preceding embodiments, further comprising collecting the multicomponent yarn onto a spool.

### Embodiment 90

A method of making a knitted article comprising:

knitting the first yarn of any of the preceding embodiments into the knitted article.

### Embodiment 91

The method of embodiment 90 further comprising:

knitting the second yarn of any of the preceding embodiments into the knitted article.

### Embodiment 92

The method of any of the preceding embodiments, wherein the second yarn comprises a secondary fiber, wherein the secondary fiber is selected from the group consisting of: rayon, acrylic, cotton, polyethylene, polypropylene, polyester, polyamide, polyurethane, spandex, silk, wool, viscose, and combinations thereof.

### Embodiment 93

The method of any of the preceding embodiments, wherein knitting the second yarn into the knitted article comprises knitting the second yarn such that the resulting knitted article has a coefficient of friction of at least 0.2 and no greater than 0.5 according to the lubricity test method.

### Embodiment 94

The method of any of the preceding embodiments, wherein knitting the second yarn into the knitted article comprises knitting a plurality of second yarns into the knitted article.

### Embodiment 94a

The method of any of the preceding embodiments, wherein knitting the first yarn into the knitted article comprises knitting a stockinette stitch.

### Embodiment 95

The method of any of the preceding embodiments, wherein knitting the first yarn into the knitted article comprises knitting a garter stitch.

### Embodiment 96

A method comprising:

applying the knitted article or the medical article to mammalian tissue.

### EXAMPLES

These examples are merely for illustrative purposes only and are not meant to be limiting on the scope of the appended claims. All parts, percentages, ratios, and the like in the examples are by weight, unless noted otherwise. Solvents and other reagents used were obtained from Sigma-Aldrich Chemical Company; Milwaukee, Wis, unless otherwise noted.

List of Materials

| Acronym | Description |
|---|---|
| TG500 | A polyether-based thermoplastic polyurethane |
| | commercially available from LUBRIZOL, |
| | Ohio, USA under the trade name |
| | TECOPHILIC Hydrogel. |
| TG2000 | A polyether-based thermoplastic polyurethane |
| | commercially available from LUBRIZOL, |
| | Ohio, USA under the trade name |
| | TECOPHILIC Hydrogel. |
| Engage 8402 | A thermoplastic polyolefin elastomer |
| | commercially available from DOW |
| | CHEMICAL, Delaware, USA. |
| Aspun 6850A | A linear low density polyethylene |
| | commercially available from DOW |
| | CHEMICAL, Delaware, USA. |
| PPS440-200 | A polyester-based thermoplastic polyurethane |
| | commercially available from HUNTSMAN, |
| | Texas, USA. |
| HP-60D-20 | A polyether-based thermoplastic polyurethane |
| | commercially available from LUBRIZOL, |
| | Ohio, USA. |
| PLA6100D | A thermoplastic resin based on Polylactic Acid |
| | commercially available from Natureworks, |
| | Minnesota, USA. |
| Cotton10 | A yarn made of 100% mercerized cotton, |
| | white, size 10 (commercially available under |
| | the trade designation Aunt Lydia's made by |
| | Coats Group plc. (UK), item 7346364). |
| Baby Yarn | A yarn made of 90% acrylic and 10% nylon |
| | fibers, superfine, white (commercially |
| | available under the trade designation Bernat, |
| | made by Spinrite Limited Partnership |
| | (Canada), item 057355340978). |
| Bamboo10 | A yarn made of 100% viscose (from bamboo), |
| | white, size 10 (commercially available under |
| | the trade designation Aunt Lydia's made by |
| | Coats Group plc. (UK), item 10147775). |

### Test Methods

### Absorption Test

49 inches of sample multicomponent yarn with 165 core-sheath multicomponent filaments were cut and weighed. For the knitted article, the multicomponent yarn and a secondary fiber (if present) were weighed separately before knitting the article. The sample multicomponent yarn or knitted article was immersed with 100 ml distilled water at 25° C. at 1 atm for 130 minutes. The distilled water was drained for 10 seconds and the wetted sample multicomponent yarn or knitted article was weighed.

### Wet Tenacity Test

The wet tenacity was measured using ISO 9073.3 tensile test method. The multicomponent yarn was immersed in approximately 100 ml distilled water for 10 minutes at ambient conditions. The distilled water was drained for 10 seconds and the tensile strength of the wetted yarn was tested using Zwick Universal Tabletop Test Model Z005 machine made by Zwick GmbH Co (Ulm, Germany). Test gauge length was 1 inch and the extension rate was 1000 mm/min.

### Lubricity Test

A knitted article was hydrated by placing the samples in an excess of distilled water for 30 minutes. The coefficient of friction of the example substrates were tested against wet sausage casings (Natural Hog Casings) (i.e., submucosa of pig intestine, obtained from The Sausage Maker, Inc., Buffalo, N.Y.). The wet sausage casing was prepared by cutting a piece of sausage casing (˜12 cm long and 3 cm wide), rinsing the sausage casing in distilled water to remove salt, and hydrating the sausage casing for at least 30 minutes in lukewarm distilled water. The sausage casing was placed on a mounting plate of a two-dimensional force testing system (under the trade designation FORCEBOARD, made by Industrial Dynamics Sweden AB (Jarfalla, Sweden)) and secured with binder clips.

Friction test substrates (i.e., sausage casing) were manually rubbed with example substrates at a target vertical force of 2.9-3.1 N. Friction coefficients were analyzed using the ForceBoard Analyzer software (Industrial Dynamics, Sweden) and recorded.

### Examples 1-7

Various multicomponent yarns comprising 165 multicomponent filaments in a sheath-core configuration were prepared using different sheath and core combinations. Examples 1-7 illustrate that core selection has an impact on the spinnability of the multicomponent yarn.

In examples 1-7 and comparative example 1, a multicomponent yarn comprising 165 multicomponent filaments in a sheath-core configuration was processed. Various sheath and core combinations of the multicomponent filament were processed. The sheath and core materials were fed in two separate extruders and coextruded at the die with a sheath/core ratio at 25/75. The core extrusion temperature varied from 195° C. to 235° C. and the sheath extrusion temperature varied from 130° C. to 230° C. A spinneret with 165 openings was used to make the sheath-core filaments with a die temperature at approximately 195° C. Some sheath and core combinations were spinnable. For the spinnable sheath-core filaments, the sheath-core filaments were collected as a tow with a winding speed of 60 to 120 meters per minute. The sheath-core filaments were not twisted. The non-spinnable sheath-core filaments were not collected. The results were collected on table 1. Unexpectedly, only select combinations of TG500 and TG2000 were spinnable. A scanning electron microscope image of example 2 is shown in FIG. 3 showing multiple sheath core fibers in a multicomponent yarn.

TABLE 1
Spinnability results of Multicomponent (MC) filaments

| | | Sheath | | Core | | |
|---|---|---|---|---|---|---|
| | | Extrusion | | Extrusion | Winding | |
| | | Temperature | | Temperature | Speed | |
| Example | Sheath | (° C.) | Core | (° C.) | (m/min) | Spinnable |
| 1 | TG500 | 180 | Engage 8402 | 195 | 60 | Yes |
| 2 | TG2000 | 130 | Engage 8402 | 195 | 120 | Yes |
| 3 | TG2000 | 130 | Aspun | 195 | 60 | Yes |
| | | | 6850A | | | |
| 4 | TG2000 | 180 | PLA6100D | 230 | Not | No |
| | | | | | Observed | |
| 5 | TG2000 | 180 | PPS440-200 | 235 | Not | No |
| | | | | | Observed | |
| 6 | 75% | 180 | PPS440-200 | 235 | Not | No |
| | TG2000; | | | | Observed | |
| | 25% HP- | | | | | |
| | 60D-20 | | | | | |
| 7 | 50% | 180 | PPS440-200 | 235 | Not | No |
| | TG2000; | | | | Observed | |
| | 50% HP- | | | | | |
| | 60D-20 | | | | | |
| CE1 | PPS440- | 230 | PLA6100D | 230 | 120 | Yes |
| | 200 | | | | | |

### Examples 8-13

In examples 8-13, multicomponent yarns with various combinations of sheath and core were spun according to the method of examples 1-7. Some of the spun multicomponent yarns were subjected to an e-beam dosage of 20 Mrads or no e-beam dosage. A line speed of 18.9 feet per minute was used with a 300 kW electron beam purged with nitrogen (oxygen less than 20 ppm). Some of the spun multicomponent yarns were also post-drawn. During post-drawing, a sample of multicomponent yarn was pulled manually. Samples with a TG2000 sheath were able to be drawn 200%-300% of the original length. A 49-inch dry sample was weighed and the results are shown in table 2. The absorption capacity was determined according to the test methods described above.

TABLE 2
Absorption capacity results of e-beam and post-drawn examples

| | | | Absorption | | |
|---|---|---|---|---|---|
| Example | Sheath | Core | capacity (%) | Post-drawn | E-beam |
| 8 | TG500 | Engage 8402 | 224 | No | Yes |
| 9 | TG2000 | Engage 8402 | 278 | No | Yes |
| 10 | TG2000 | Engage 8402 | 318 | Yes | Yes |
| 11 | TG500 | Engage 8402 | 679 | No | No |
| 12 | TG2000 | Engage 8402 | 996 | No | No |
| 13 | TG2000 | Engage 8402 | 960 | Yes | No |

### Examples 14-20

Examples 14-20 were prepared using the composition and method of example 2 described above. Dosage levels of electron beam radiation were varied according to table 3. A 300 kW electron beam machine was used with a line speed of 18.9 feet per minute. The ebeam machine was purged with nitrogen under standard temperature and pressure conditions. The oxygen level was less than 20 ppm.

TABLE 3
Absorption and wet tensile strength results for various e-beam dosages

| Example | E-beam dosage | Absorption (g/g) | Wet Tensile Strength (N) |
|---|---|---|---|
| 14 | 1 | 3.9 | 16.9 |
| 15 | 3 | 4.39 | 21 |
| 16 | 5 | 3.9 | 20.3 |
| 17 | 10 | 3.11 | 22 |
| 18 | 15 | 2.02 | 26.6 |
| 19 | 20 | 1.64 | 24.3 |
| 20 | 0 | 4.05 | 16.7 |

### Examples 21-27

Examples 21-27 and comparative example 2 were prepared by forming a knitted article of at least the multicomponent yarn from example 1 and either bamboo10 yarn or baby yarn. Comparative example 10 was made with only Bamboo10 yarn. During post-drawing, a sample of multicomponent yarn was pulled manually. Samples with a TG500 sheath were able to be drawn 300%-400% of the original length. A size 7 needle was used. The dry weight of the multicomponent yarn and secondary fiber (in the form of a second yarn) was recorded. The multicomponent yarn was knitted using the stitch and the secondary fiber was threaded through (unlinked) the finished knitted article noted in table 4.

TABLE 4
Composition of knitted articles comprising TG500 multicomponent (MC) yarn

| | | | | | | | Number of |
|---|---|---|---|---|---|---|---|
| | Wt. MC | MC Yarn | 2^{nd} Fiber | % MC | % 2^{nd} | | strands of |
| Example | Yarn (g) | Size (tex) | Wt. (g) | yarn | fiber | Stitch Type | 2^{nd} yarns |
| 21⁺ | 0.6382 | 213 | | 100% | | Garter | 0 |
| 22⁺ | 0.8607 | 287 | 0.1444** | 86% | 14%** | Stockinette | 1 |
| 23 | 1.31 | 437 | 0.46* | 74% | 26%* | Stockinette | 1 |
| 24 | 1.7 | 567 | 1.06* | 62% | 38%* | Stockinette | 2 |
| 25 | 0.8435 | 281 | 0.823** | 51% | 49%** | Garter | 1 |
| 26 | 1.61 | 537 | 1.61* | 50% | 50%* | Stockinette | 3 |
| 27 | 1.24 | 413 | 1.5* | 45% | 55%* | Stockinette | 3 |
| CE2 | | | 2.15* | 0% | 100%* | Stockinette | 3 |

*indicates Bamboo 10
**indicates Baby Yarn
⁺indicates sample was post-drawn

The absorption and wet friction of Examples 21-27 and CE2 were determined using the test methods described above and recorded in table 5.

TABLE 5
Absorption capacity and wet friction results of knitted articles

Wet Friction

| Example | Absorption (%) | Average | Standard Dev. |
|---|---|---|---|
| 21 | 548% | 0.26 | 0.03 |
| 22 | 531% | 0.48 | 0.08 |
| 23 | 339% | 0.31 | 0.03 |
| 24 | 256% | 0.41 | 0.02 |
| 25 | 321% | 0.43 | 0.03 |
| 26 | 249% | 0.33 | 0.03 |
| 27 | 267% | 0.37 | 0.03 |
| CE2 | 281% | 0.72 | 0.04 |

### Examples 28-32

Examples 28-32 and comparative example 3 were prepared by forming a knitted article of at least the multicomponent yarn from example 2 and either bamboo10 yarn, cotton10, or baby yarn. During post-drawing, a sample of multicomponent yarn was pulled manually. Samples with a TG2000 sheath were able to be drawn 200%-300% of the original length. A size 7 needle was used to form the article. The dry weight of the multicomponent yarn and secondary fiber (in the form of a second yarn) was recorded. The multicomponent yarn was knitted using the stitch and the secondary fiber was threaded through (unlinked) the finished knitted article noted in table 6.

TABLE 6
Composition of knitted articles comprising TG2000 multicomponent (MC) yarn

| | | | | | | | Number |
|---|---|---|---|---|---|---|---|
| | | | | | | | of |
| | | MC Yarn | | | | | strands |
| | Wt. MC | Size | 2^{nd} Fiber | % MC | % 2^{nd} | Stitch | of 2^{nd} |
| Example | Yarn (g) | (tex) | Wt. (g) | yarn | fiber | Type | yarns |
| 28⁺ | 0.12 | | 1.4616*** | 8% | 92%*** | Stockinette | 2 |
| 29⁺ | 1.1 | 367 | 1.49* | 42% | 58%* | Stockinette | 3 |
| 30 | 0.886 | 295 | 0.97** | 48% | 52%** | Stockinette | 3 |
| 31 | 1.04 | 347 | 1.00* | 51% | 49%* | Stockinette | 2 |
| 32 | 0.96 | 320 | 0.50* | 66% | 34%* | Stockinette | 1 |
| CE3 | 0.98 | 327 | | 100% | 0% | Stockinette | 0 |

*indicates Bamboo 10
**indicates Baby Yarn
***indicates Cotton 10
⁺indicates post-drawn

The absorption and wet friction of Examples 28-32 and CE3 were determined using the test methods described above and recorded in table 7.

TABLE 7
Absorption capacity and wet friction results of knitted articles

Wet Friction

| Example | Absorption (%) | Average | Standard Dev |
|---|---|---|---|
| 28 | 300% | 0.49 | 0.03 |
| 29 | 286% | 0.46 | 0.04 |
| 30 | 328% | 0.37 | 0.03 |
| 31 | 330% | 0.33 | 0.03 |
| 32 | 391% | 0.25 | 0.05 |
| CE3 | 618% | 0.07 | 0.02 |

As can be shown above, properties of the knitted article can be tuned using secondary fibers and/or processing techniques.

A woven example was also prepared from the multicomponent yarn.

1. A multicomponent filament, comprising:
a first component comprising a hydrophobic thermoplastic polymer; and
a second component comprising a hydrophilic thermoplastic polymer comprising 65% (w/w) to 90% (w/w), inclusive, hydrophilic segments;
wherein the first component is capable of forming a continuous filament with the second component.

2. The multicomponent filament of claim 1, wherein the thermoplastic polymer comprises a thermoplastic olefin having a melt flow index from 10 g/10 min to 100 g/10 min at 190° C. (inclusive).

3. The multicomponent filament of claim 2, wherein the hydrophilic segments comprise a polyalkylene oxide.

4. The multicomponent filament of claim 3, wherein the hydrophilic segments are selected from the group consisting of polyethylene glycol, polypropylene glycol, polybutylene oxide, random poly(C2-C4)alkylene oxide, polyester, amine-terminated polyester, amine-terminated polyamide, polyester-amide, polycarbonate, and combinations thereof.

5. The multicomponent filament of claim 2, wherein the first component is a core and second component is a sheath in a core/sheath multicomponent filament.

6. A first yarn comprising the multicomponent filament of claim 1.

7. An article comprising the first yarn of claim 6, wherein the article is selected from a group consisting of a knitted article, a woven article, nonwoven article, and combinations thereof.

8. The article of claim 7, further comprising a secondary fiber wherein the secondary fiber is selected from the group consisting of: rayon, cotton, polyethylene, polypropylene, polyester, nylon, or combinations thereof.

9. The article of claim 8, further comprising a second yarn comprising the secondary fiber.

10. The article of claim 9, wherein the article includes enough second yarn such that a coefficient of friction of the article is at least 0.2 and no greater than 0.5 according to a lubricity test method.

11. A medical article having the article of any of claim 7 disposed thereon.

12. A method of making the multicomponent filament of any of claim 1, comprising:
extruding, through a die, the first component at a first temperature, and the second component at a second temperature, to form a multicomponent filament.

13. The method of claim 12, further comprising:
drawing the multicomponent filament; and
processing a plurality of the multicomponent filaments into a multicomponent yarn.

14. The method of claim 13, further comprising:
crosslinking at least one portion of a multicomponent filament from the plurality of multicomponent filaments.

15. A method of making the article of claim 7 comprising:
forming the article from the first yarn;
adding the second yarn into article such that a resulting article has a coefficient of friction of at least 0.2 and no greater than 0.5 according to the lubricity test method.

16. The method of claim 12, wherein a difference between the first temperature and the second temperature is at least 5 degrees Celsius, with the first temperature being higher than the second temperature.

17. The method of claim 12, wherein the crosslinking occurs such that the multicomponent filament exhibits water absorption no greater than 6 grams water per gram multicomponent filament according to the Absorption Test Method.

18. The multicomponent filament of claim 1, wherein the multicomponent filament has an average diameter of no greater than 50 micrometers.

19. The multicomponent filament of claim 1, wherein the hydrophilic thermoplastic polymer is an aliphatic thermoplastic polyurethane (TPU) polymer.

20. The multicomponent filament of claim 3, wherein block subunits of polyalkylene oxide have a formula weight of between 1000 and 2000 daltons (inclusive).
